(19) United States
(12) Patent Application Publication
(10) Pub. No.: US 2026/0241201 A1
(43) Pub. Date: Aug. 20, 2026

(54) AFTERLOADING THERAPY DEVICE

(71) Applicants: Jianrong Dai, Beijing (CN); Chuanmeng Niu, Beijing (CN)

(72) Inventors: Jianrong Dai, Beijing (CN); Chuanmeng Niu, Beijing (CN)

(21) Appl. No.: 19/345,788

(22) Filed: Sep. 30, 2025

(30) Foreign Application Priority Data
Feb. 14, 2025 (CN) 202510165467.X

(51) Int. Cl. A61N 5/10

(52) U.S. Cl. CPC A61N 5/1007; A61N 2005/1008

(57) ABSTRACT

An afterloading therapy device is provided, which comprises: a base; a plurality of source modules for placing active or dummy sources; a rotating assembly installed on the base and comprising a rotating frame, the rotating frame being equipped with a plurality of source module installation positions, and the source modules being configured to be rotatably installed on the base through the rotating assembly; and an indexing assembly comprising an indexing plate and a plurality of indexing holes which are arranged on the indexing plate and corresponding to the plurality of source modules; wherein, when observing along the source application path of the active or dummy sources, the indexing assembly is arranged on the downstream side of the rotating assembly. Through this configuration, it is possible to improve the performance of the afterloading therapy device by arranging a plurality of source modules.

### TECHNICAL FIELD

The present invention relates to the field of medical device technology, and in particular provides an afterloading therapy device.

### BACKGROUND

As a radiation source driving device, the working principle of an afterloading therapy machine is to transport the radiation source to the dwell position located near the malignant tumors within a patient's cavity, lumen or tissue, thereby enabling localized radiation treatment for the lesion. After completing the therapy, the radiation source is retracted back into a shielded container within the device.

Currently, most afterloading therapy machines are configured with only a single active radiation source. In such a design, each insertion cycle allows radiation to be delivered to only one dwell position. Consequently, treatment time increases proportionally with the number of dwell positions. For cases involving multiple dwell positions within the tumor region, an indexing mechanism is typically used to sequentially direct the radiation source to each position. Prior to treatment, a dummy source must verify the patency of the delivery channels. Furthermore, as the radioactivity of the source decays over time, treatment duration extends even further. To maintain therapeutic efficiency, partially decayed sources (e.g., at half-life) are often replaced prematurely, resulting in unnecessary source waste and increased disposal costs.

Accordingly, there is a need for an improved technological solution that addresses the above-described limitations in current afterloading therapy devices.

### SUMMARY

The present invention aims to at least partially address the technical problems described above. In particular, the present invention provides an afterloading therapy device that allows flexibly configuration of one or more source modules based on the needs of a patient population. For each individual patient, one or more source modules may be selectively activated, either in parallel or sequentially, depending on the plan. This configuration not only shortens treatment duration and improves operational efficiency, but also extends the usable life of each radioactive source and reduces the frequency and cost of source replacement.

In view of this, the present invention provides an afterloading therapy device, which comprises: a base; a plurality of source modules for placing active or dummy sources; a rotating assembly which is installed on the base and comprises a rotating frame, the rotating frame being equipped with a plurality of source module installation positions thereon, and the source modules being configured to be rotatably installed on the base through the rotating assembly; and an indexing assembly comprising an indexing plate and a plurality of indexing holes arranged on the indexing plate and corresponding to the plurality of source modules; wherein, when observing along a source application path of the active or dummy source, the indexing assembly is arranged on a downstream side of the rotating assembly.

With this configuration, it is possible to improve the performance of the afterloading therapy device by arranging a plurality of source modules. Moreover, as only one indexing assembly is comprised, it further improves the performance of the afterloading therapy device.

It can be understood that a person skilled in the art can determine the structural form, number, relative position, etc. of the source module depending on actual needs. For example, the structures of the plurality of source modules can be the same as or different from each other, and they can be evenly or unevenly distributed at different positions on the rotating frame.

As for the above mentioned afterloading therapy device, in one possible implementation, the afterloading therapy device further comprises: a convergence assembly comprising a convergence layer and a plurality of convergence pipes which are arranged on the convergence layer and corresponding to the plurality of indexing holes; and a connecting assembly comprising a pipe connecting plate bracket and a plurality of pipe connecting plate holes which are arranged on the pipe connecting plate bracket and corresponding to the plurality of convergence pipes; wherein, the convergence assembly is located between the indexing assembly and the pipe connecting assembly.

With this configuration, possible structural forms of the afterloading therapy device are provided.

As for the above mentioned afterloading therapy device, in one possible implementation, the rotating assembly comprises a driving part, wherein the driving part comprises: a driving component; and a support shaft arranged on the base. The driving component can drive the rotating frame to rotate through the support shaft and thus drive the source module arranged on the rotating frame to move.

With this structure, it is possible to achieve the movement of the source module based on the driving force provided by the driving components, which may be such as a motor or any form of rotating module (such as a combination of motor and gear pair).

As for the above mentioned afterloading therapy device, in one possible implementation, the rotating frame is implemented as an annular (ring-shaped) structure, and the driving component can drive the rotating frame to rotate around its axis.

With this configuration, it is possible to achieve the source module being placed in the working position by rotation of the rotating frame.

As for the above mentioned afterloading therapy device, in one possible embodiment, the rotating frame comprises one or more ring-shaped structures distributed radially, one or more support shafts corresponding to the rotating frames are fixedly arranged on the base, and one or more driving components corresponding to the one or more rotating frames can drive a plurality of rotating frames to rotate relative to their respective support shafts in an independent manner.

With this configuration, it is possible to better meet the performance requirements of the afterloading therapy device through one or more rotating frames and provide possible driving connection modes between the driving components and a plurality of rotating frames.

As for the above mentioned afterloading therapy device, in one possible implementation, the support shaft has a cylindrical structure, the rotating frame is arranged on an outer wall of the support shaft, and the driving part further comprises a transmission mechanism, which comprises a gear connected to a power output end of the driving component, and a gear ring which is arranged on an inner or outer wall of the cylindrical structure. The gear can be meshed with the gear ring, and thus enable the driving component to rotatably connect with the support shaft through the transmission mechanism.

With this configuration, it is possible to achieve connecting the driving component to the rotating frame through the transmission mechanism. In the case where the rotating frame comprises one layer, the inner wall of the cylindrical structure is provided with a gear ring, and in the case where the rotating frame comprises a plurality of layers, the outer wall of the cylindrical structure is provided with a gear ring.

As for the above mentioned afterloading therapy device, in one possible implementation, the rotating frame comprises one or more ring-shaped structures distributed radially, and the support shaft is fixedly arranged on the rotating frame. In the case where there is one ring-shaped structure, the driving component can drive the support shaft to rotate synchronously with the rotating frame; in the case where there are a plurality of ring-shaped structures, the driving component can drive the support shaft to rotate synchronously with the plurality of ring-shaped structures.

With this structure, it is possible to better meet the performance requirements of the afterloading therapy device and provide possible driving connection modes between the driving components and multiple rotating frames (the driving components drive multiple rotating frames to rotate relatively independently) by multiple rotating frames. As an example, in the case of a rotating frame where each layer in a single or multiple layers can independently rotate, the support shaft is a cylindrical structure fixed on the base. In the case of synchronous rotation of single-layer or multi-layers of the rotating frame, the support shaft is a solid shaft fixed to the rotating frame.

As for the above mentioned afterloading therapy device, in one possible implementation, the rotating frame comprises one or multiple ring-shaped structures distributed radially. In the case where there are multiple rotating frames, the indexing plate is provided with a plurality sets of indexing holes corresponding to the multiple ring-shaped structures, the convergence layer is provided with a plurality sets of convergence pipes corresponding to the multiple ring-shaped structures, and the pipe connecting plate bracket is provided with a plurality sets of pipe connecting plate holes corresponding to the multiple ring-shaped structures.

With this configuration, possible structural forms of the afterloading therapy device are provided in the case where a plurality of rotating frame are comprised.

For the above mentioned afterloading therapy device, in one possible implementation, the source module can be arranged in a detachable manner at the source module installation position on the rotating frame.

With such a configuration, it is possible to improve the maintenance efficiency of the source module.

As for the above mentioned afterloading therapy device, in one possible implementation, the rotating frame is provided with a first sliding structure at a position corresponding to the installation position of the source module, and the source module comprises: a support structure on which a second sliding structure is provided; and a source storage tank that can provide active or dummy sources; wherein the source module can be arranged in a push-pull manner at the installation position of the source module by means of the sliding connection of the first sliding structure and the second sliding structure.

With this configuration, possible implementation methods for sliding connections in detachable connections are provided.

In the preferred embodiments of the present invention, one or more rotating frames containing a plurality of source module installation positions are used. On this basis, a plurality of source modules can be configured on the afterloading therapy device depending on actual needs. Therefore, the usage efficiency of the source modules can be improved, the cost of source replacements can be reduced, and a plurality of source modules can be used simultaneously for brachytherapy, thereby reducing therapy time. In addition, only one indexing assembly is comprised, and the source module can move either synchronously or relatively independently, making the device more flexible in use.

### BRIEF DESCRIPTION OF THE DRAWINGS

In order to provide a clearer explanation of the technical solution of the embodiments of the present invention, a brief introduction will be given to the accompanying drawings required for the description of the embodiments. It is obvious that the drawings described below are only some embodiments of the present invention. For those skilled in the art, other drawings can be obtained based on these drawings without creative labor.

FIG. 1 shows a schematic diagram I of the structure of the afterloading therapy device using a single rotating assembly according to a first embodiment of the present invention;

FIG. 2 shows a schematic diagram II of the structure of the afterloading therapy device of the first embodiment of the present invention without installing the source module, with the source module removed from the figure;

FIG. 3 shows a schematic diagram III of the structure of the indexing assembly of the afterloading therapy device according to the first embodiment of the present invention, in which the indexing hole, the convergence pipe, and the pipe connecting plate hole are shown in perspective;

FIG. 4 shows a schematic diagram IV (front view) of the structure of the afterloading therapy device according to the first embodiment of the present invention;

FIG. 5 shows a schematic diagram of the structure of the source module in the afterloading therapy device according to an embodiment of the present invention;

FIG. 6 shows a schematic diagram of the principle of the afterloading therapy device according to an embodiment of the present invention;

FIG. 7 shows a schematic diagram I of the structure of the afterloading therapy device according to a second embodiment of the present invention (three-dimensional);

FIG. 8 shows a schematic diagram II (rear view) of the structure of the afterloading therapy device according to the second embodiment of the present invention;

FIG. 9 shows a schematic diagram III (front view) of the structure of the afterloading therapy device according to the second embodiment of the present invention;

FIG. 10 shows a schematic diagram of the structure of the afterloading therapy device according to the second embodiment of the present invention without installing the source module;

FIG. 11 shows a schematic diagram of the structure of the support shaft in the afterloading therapy device according to the second embodiment of the present invention;

FIG. 12 shows a schematic diagram of the structure of the afterloading therapy device according to a third embodiment of the present invention;

FIG. 13 shows a schematic diagram of the structure of the afterloading therapy device of the third embodiment of the present invention without installing the source module;

FIG. 14 shows a schematic diagram of the structure of the rotating frame in the afterloading therapy device of the third embodiment of the present invention;

FIG. 15 shows a schematic diagram I of the structure of the afterloading therapy device according to a fourth embodiment of the present invention;

FIG. 16 shows a schematic diagram II of the structure of the afterloading therapy device according to the fourth embodiment of the present invention, in which the indexing hole is shown.

In the figures:
100. afterloading therapy device;
1. base;
2. source module;
3. rotating assembly;
4. indexing component;
5. convergence assembly;
6. pipe connection plate assembly;
7. source applicator.

### DETAILED DESCRIPTION

The preferred embodiments of the present invention will be described below with reference to the accompanying drawings. A person skilled in the art should understand that these embodiments are only used to explain the technical principles of the present invention and are not intended to limit the scope of protection of the present invention in any way.

The following provides a detailed description of the embodiments of the present invention, and throughout the disclosure, same or similar reference numerals represent same or similar elements or elements with the same or similar functions. The embodiments described below with reference to the accompanying drawings are exemplary and only used to explain the present invention, and cannot be interpreted as limiting the present invention.

Those skilled in the art can understand that, unless specifically stated, the singular forms such as “one”, “a/an”, “the”, and “said” used here may also cover plural forms. It should be further understood that the term “comprising” used in the specification of the present invention refers to the presence of the described features, integers, steps, operations, elements, and/or components, but does not exclude the presence or addition of one or more other features, integers, steps, operations, elements, components, and/or combinations thereof. It should be understood that when referring to a component as being “connected” or “coupled” to another component, it can be directly connected or coupled to other components, or there may be intermediate components present. In addition, the term “connected” or “coupled” used here may include wireless connection or coupling. The wording “and/or” used here comprises any unit listed in one or more associated listed items and all combinations thereof.

Those skilled in the art can understand that, unless otherwise defined, all terms used herein (comprising technical terms and scientific terms) have the same meaning as those generally understood by a person skilled in the art. It should also be understood that terms such as those defined in general dictionaries should be understood to have meanings consistent with those in the context of prior art, and unless defined as such herein, they will not be interpreted with idealized or overly formal meanings.

In addition, in order to better illustrate the present invention, numerous specific details are provided in the following specific embodiments. Those skilled in the art should understand that the present invention can also be implemented without certain specific details. In some examples, the principles such as those of the source modules that are well-known to those skilled in the art have not been described in detail in order to highlight the main idea of the present invention.

For facilitating understanding the embodiments of the present invention, several specific embodiments will be further explained and illustrated with reference to the accompanying drawings, and each embodiment does not cause any limitation on the embodiments of the present invention in any manner.

### Embodiment 1

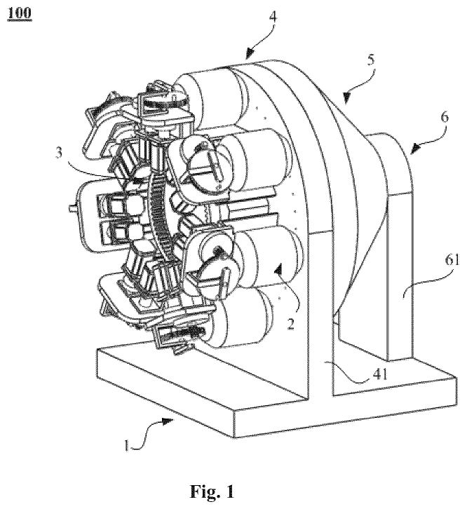

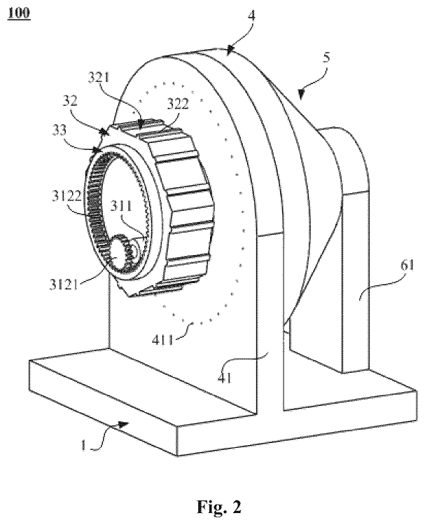

Referring mainly to FIGS. 1 to 6, in one possible embodiment, the afterloading therapy device 100 of the present invention mainly comprises a base 1, a source module 2, a rotating assembly 3, an indexing assembly 4, a convergence assembly 5, a pipe connecting plate assembly 6, and a source applicator 7. The base 1 mainly serves as the installation carrier for the indexing assembly 4 and the pipe connecting plate 5. The source module 2 can provide active or dummy sources, and one or more source modules 2 are arranged on the base through the rotating assembly 3. The rotating assembly is mainly used to align the source module 2 with the indexing assembly 4. The indexing assembly 4, the convergence assembly 5, and the pipe connecting plate assembly 6 are mainly used to cooperate to construct a path through which the active or dummy source can accurately reach the source applicator source 7.

In one possible implementation, an indexing plate 41 and a pipe connecting plate bracket 51 are provided on the base 1, the rotating assembly 3 is provided on the first side (left side, back in FIG. 1) of the indexing plate 41, one or more source modules 2 can be provided on the base through the rotating assembly, and the convergence layer of the indexing component 4 is provided on the second side (right side, front in FIG. 1) of the indexing plate 41. Obviously, a person skilled in the art can determine the structural form of the base depending on actual needs, as long as installation space for corresponding components on the, for example, integrally formed irregular structure could be reserved.

In one possible embodiment, the rotating assembly 3 mainly comprises a driving part, a rotating frame 32, and a support shaft 33. In this embodiment, the support shaft 33 is fixedly arranged on the base 1. For example, the support shaft is a hollow cylindrical structure, and the rotating frame 32 is rotatably arranged on the support shaft 33. The driving part can drive the rotating frame to rotate relative to the support shaft and the base.

In one possible implementation, the driving part mainly comprises a driving component (actuator) 311 and a transmission mechanism. The driving component is connected to the rotating frame through the transmission mechanism, thereby driving the rotating frame to rotate relative to the base. For example, the driving component can be fixedly arranged on the rotating frame or the support shaft, and correspondingly, the transmission mechanism is arranged on the support shaft or the rotating frame. The driving components can be driving motors, power cylinders (such as hydraulic cylinders, air cylinders, electric cylinders, etc.), rotating modules (such as combinations of motors and gear pairs), and the transmission mechanism can be gear pairs, belt transmission mechanism, connecting rod components, etc. In this example, the driving component is installed on the support shaft, and the transmission mechanism is a gear pair, which comprises a gear 3121 and a gear ring 3122. The power output shaft of the driving component is connected to the gear, and the gear ring is installed on the inner wall of the support shaft. The gear can mesh with the inner side of the gear ring.

In one possible implementation, the rotating frame 32 is substantially a ring-shaped structure, and a plurality of source module installation positions 321 are provided along its circumferential direction on the rear surface of the ring-shaped structure. The source modules can be detachably installed at the source module installation positions. In this example, the amount of the source module installation positions 321 is 7. Obviously, those skilled in the art can determine the structural form, number, and distribution of the source module installation positions depending on actual needs.

In one possible implementation, the source module can be arranged on the installation position in a push-pull manner. In this way, when the source module needs to be repaired, it can be pulled out between the source modules. After the repair is completed, the source modules can be directly pushed in and locked, making the operation convenient. A first sliding structure 322 for example is arranged at the position corresponding to the installation position of each source module, and a second sliding structure that can match and connect with the first sliding structure is arranged on the source module. In this way, the push-pull operation of the source module can be achieved through the cooperation of the first sliding structure and the second sliding structure. In this example, the first sliding structure is a double slide rail arranged on the installation position, and the second sliding structure can be a double slide path matched with the double slide rail. Obviously, a person skilled in the art can determine the specific form of the first/second sliding structure depending on actual needs, such as swapping the two, arranging the slide rail as a sliding rail pair along a vertical plane (the second sliding structure is located on the side of the source module), combining a slide block with the slide rail, and combining the slide rail with a shaft. In addition, besides being push-pull, the source module can also be assembled or disassembled onto/from the rotating frame through other methods such as buckling engagement and engagement/disengagement switching with the help of clutch devices.

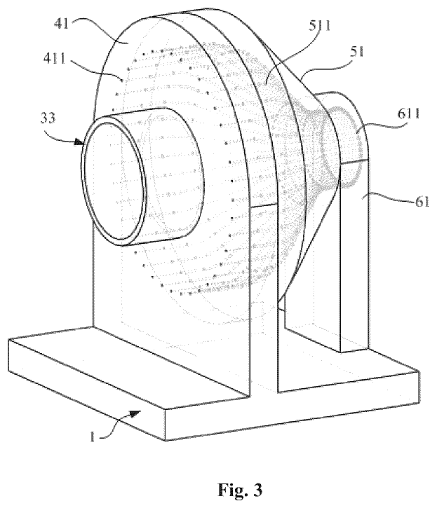

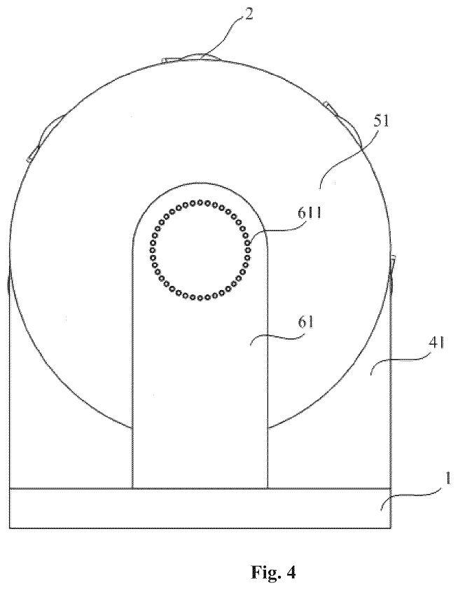

Referring mainly to FIGS. 3 and 4, in one possible embodiment, the indexing assembly 4 mainly comprises an indexing plate 41, which is arranged on the base and has through indexing holes 411 along its circumference. The convergence assembly 5 mainly comprises a convergence layer 51, which contains convergence pipes 511 in an amount equal to that of indexing holes on the indexing plate. The pipe connecting plate assembly 6 comprises a pipe connecting plate 61 and a pipe connecting plate bracket 62. The pipe connecting plate 61 is arranged on the base through the pipe connecting plate bracket 62, and the pipe connecting plate 51 is arranged at the end of the convergence layer (on the side distal away from the indexing plate). The pipe connecting plate 61 is provided with pipe connecting plate holes 611 in an amount equal to the number of convergence pipes. The supporting shaft 33 of the cylindrical structure is fixedly arranged on a first side (left side) of the indexing plate. Under the driving of the rotating assembly 3, the output hole on the source module 2 can be aligned with the indexing hole coaxially. In this embodiment, since the rotating frame is a ring-shaped structure, the indexing holes 411, the convergence pipes 511, and the pipe connecting plate holes 611 all include a group distributed in a roughly ring-shaped structure.

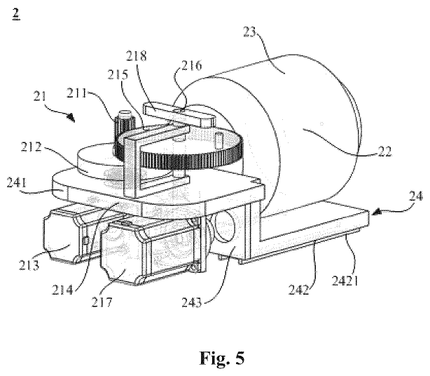

Referring mainly to FIG. 5, in one possible implementation, the source module 2 mainly comprises a cable winding mechanism 21, a source cable 22 carrying a radiation source 221, a source storage tank 23, and a support structure 24. The source module can accommodate both source cables with real and dummy sources. In this example, the supporting structure is substantially a Z-shaped structure (mainly comprising a first horizontal part 241 above, a second horizontal part 242 below, and a vertical part 243 connecting the two). A second sliding structure 2421 is provided below the second horizontal part, such as a downward protruding track. The cable winding mechanism is located on the first side of the vertical part (left side in FIG. 5) and installed on the first horizontal part. The source storage tank is installed on the second side of the vertical part (right side in FIG. 5), and there is a through-hole for the source cable to enter and exit on the source storage tank. The source cable is arranged on the cable winding mechanism, and under the drive of the cable winding mechanism, the end of the source cable can be retracted to the storage tank or protrude out the storage tank to a certain length outside.

In this example, the cable winding mechanism 21 mainly comprises a wheel shaft 211, a cable pulley 212, a cable winding motor 213, a cable winding transmission mechanism 214, an emergency return transmission mechanism 215, an emergency return shaft 216, an emergency return motor 217, and a manual return handle 218. The wheel shaft is rotatably arranged on the first transverse part of the support plate, and the part of the wheel shaft located above the first transverse part is sequentially provided with a cable pulley and an emergency return transmission for winding the source cable from bottom to top. The part of the wheel shaft located below the first transverse part is connected to the cable winding motor through the cable winding transmission mechanism. The cable winding motor for example can be installed on the support structure through a motor bracket. The part of the emergency return shaft located above the first transverse part is equipped with a limit frame, an emergency return transmission and a manual return handle, and the part of the emergency return shaft located below the first transverse part is connected to the emergency return motor through a clutch and a coupling. The emergency return motor for example can be installed on the support plate through a motor bracket, and the clutch is arranged between the emergency return motor and the emergency return transmission mechanism. In this example, both ends of the U-shaped limit frame are installed on the emergency return shaft, which can limit the rotation range of the cable winding mechanism. In this example, the cable winding motor drives the cable wheel to rotate through the wheel shaft, and the small gear above the cable wheel drives the large gear to rotate. There is a stopper on the large gear. When the source cable is just recovered to a certain position in the source storage tank, the stopper comes into be just in contact with the limit frame, thus preventing the motor from accidentally pulling the radiation source to the cable wheel. Based on this, emergency source recovery can include two methods: one is to control (such as remote control) the emergency source recovery motor, which drives the source wheel to rotate through the emergency source recovery transmission structure, wraps the source cable, and recovers the radiation source into the source storage tank. Specifically, when an emergency return is required, the clutch engages the emergency return motor with the emergency return transmission mechanism, thereby transmitting the power of the emergency return motor to the emergency return shaft. In this way, the cable wheel is driven to rotate through gear transmission, and therefore the radiation source can be recovered into the source storage tank. Another method is to manually rotate the manual return handle, which drives the source wheel to rotate through the emergency return transmission mechanism, wraps the source cable, and recovers the radiation source into the source storage tank. Obviously, this is just an exemplary description of the source module, and a person skilled in the art can determine the specific form of the source module depending on actual needs.

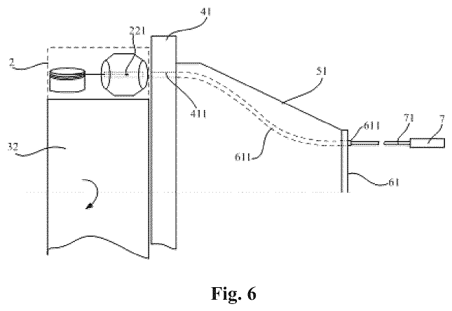

Referring mainly to FIG. 6, the working principle of the afterloading therapy device 100 is that the driving part of the rotating assembly drives the rotating frame 32 to rotate, thereby aligning each source module 2 with the corresponding indexing hole 411 on the indexing plate 41. Based on this, the radiation source (active or dummy) 221 can be controlled to reach the source applicator 7 through the arranged indexing hole 411 according to the plan. Specifically, the cable winding motor drives the cable wheel to rotate, so that the active or dummy source protrudes out of the storage tank under the drive of the source cable, and sequentially passes through the indexing hole 411, the convergence pipe 511, the pipe connecting plate hole 611, and the source conduit 71 to reach the source applicator 7, thereby achieving the corresponding irradiation position for this therapy.

### Embodiment 2

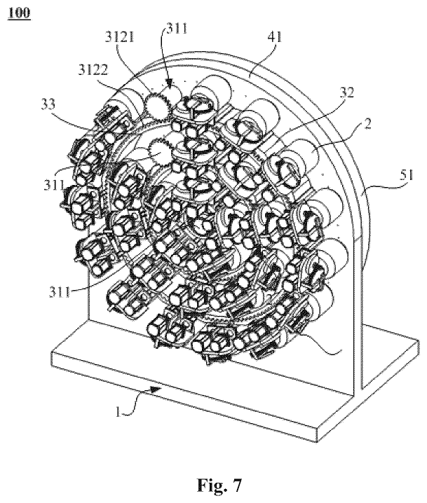

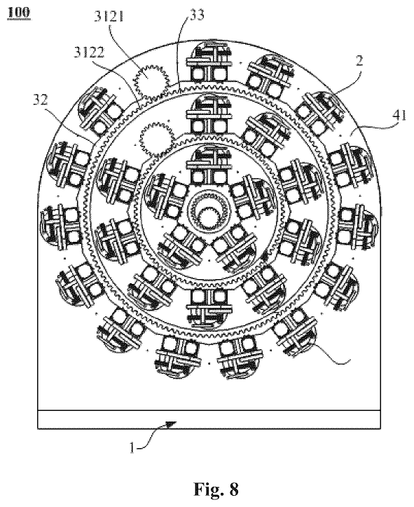

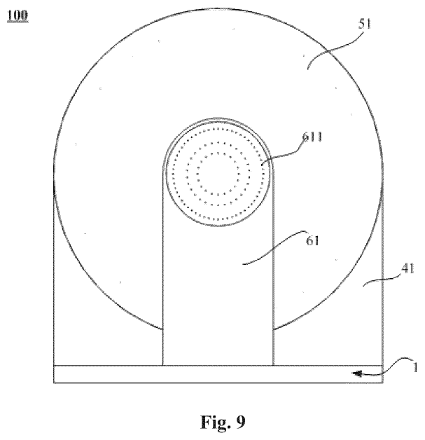

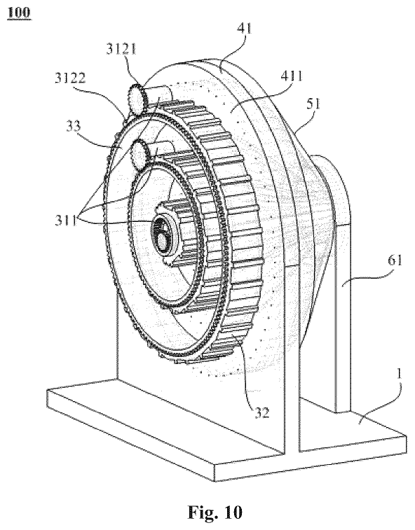

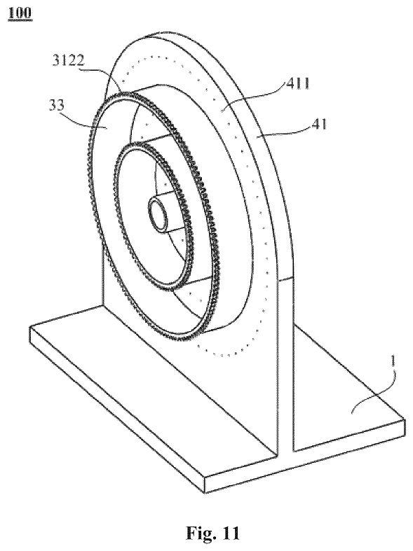

Referring mainly to FIGS. 7 to 11, in this embodiment, there are three rotating frames 32, which are provided on the support shaft 33 of three cylindrical structures arranged in layers from the inside out along the circumference, and each rotating frame is equipped with an independent driving part. In this example, the driving component 311 of the driving part corresponding to the rotating frames at the outermost and middle layers can be installed on one of the source module installation positions, and the driving component 311 of the driving part corresponding to the rotating frame at the innermost layer can be installed on the inner wall of the support shaft 33. In this example, the transmission mechanism is a gear pair, and the power output shaft of the driving component is connected to the gear 3121 of the gear pair. The gear ring 3122 of the gear pair is installed on the outer wall of the support shaft, and the gear can mesh with the outer side of the gear ring. Correspondingly, the indexing plate 41 is equipped with three-layer indexing holes 411 distributed radially thereon, the convergence layer 51 is equipped with three-layer convergence pipes 511 distributed radially thereon, and the pipe connecting plate 61 is equipped with three-layer pipe connecting plate holes 611 distributed radially thereon. The other structures such as the source module 2 are similar to those in the first embodiment, and will not be repeated here.

### Embodiment 3

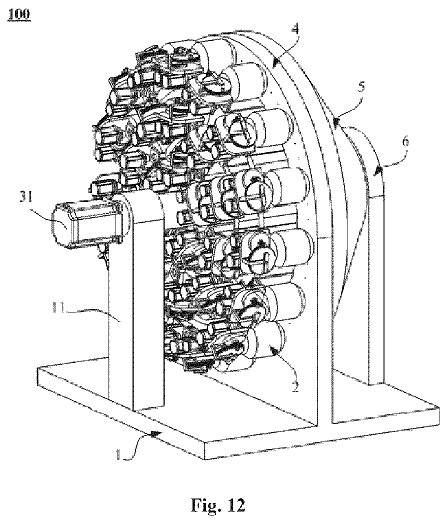

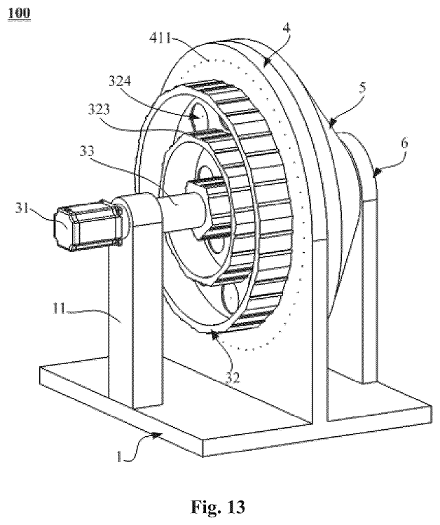

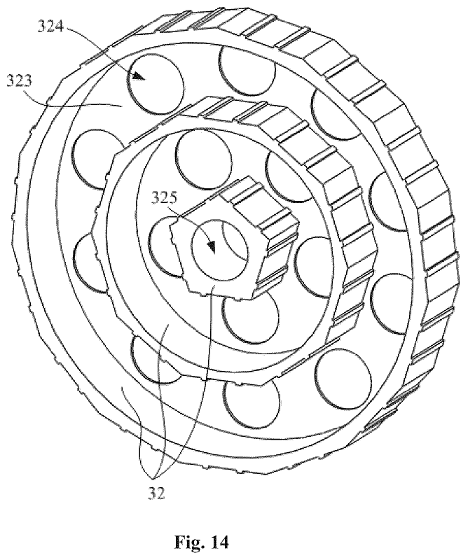

Referring mainly to FIGS. 12 to 14, in this embodiment, the rotating frames 32 arranged in layers along the radial direction are similar to those of Embodiment 2, except that the multi-layer rotating frames are fixedly connected or integrally formed. In this embodiment, the multi-layer rotating frames are connected to each other through a plate-shaped connection part 323, and a through-hole 324 is provided on the connection part corresponding to the position of the source module to allow the source cable to pass through. The multi-layer rotating frame 32 shares a common driving part. In this example, the rotating frame at the innermost layer in the radial direction has five source module installation positions, the middle layer has ten source module installation positions, and the rotating frame at the outermost layer in the radial direction has fifteen source module installation positions. The base 1 is provided with a vertical column 11, and the driving component 311 is installed on the vertical column 11. The power output end of the driving component 311 is connected to the support shaft 33, which is pivotally arranged on the indexing plate 41, and the support shaft 33 is fixedly connected to the combination of a plurality of rotating frames 32. In this example, the support shaft 33 is fixedly connected to the rotating frame 32 at radially innermost layer. For example, the rotating frame at the innermost layer in the radial direction is roughly a cylindrical structure, and the center of the cylindrical structure has a shaft hole 325 that can be matched with the support shaft 33.

### Embodiment 4

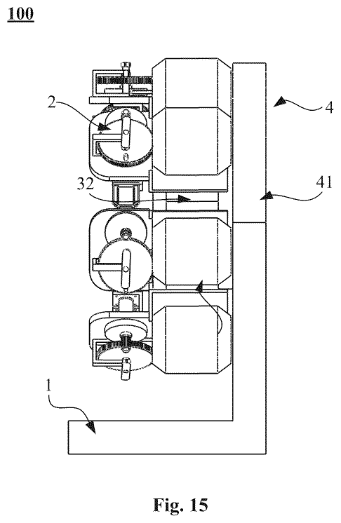

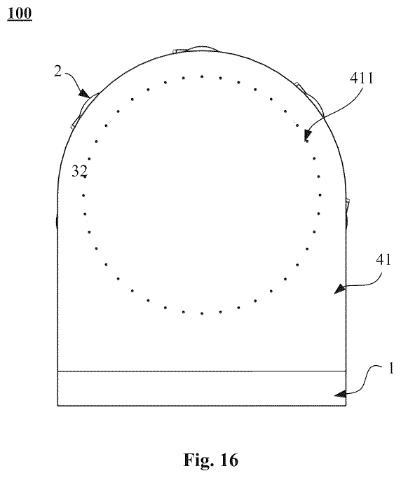

Referring mainly to FIGS. 15 and 16, unlike embodiment 1 described above, in this embodiment, the convergence assembly 5 and the pipe connecting assembly 6 between the downstream side of the indexing assembly 4 and the source applicator 7 are omitted. By directly connecting the upstream end of the source conduit 71 to the indexing hole 411 of the indexing assembly 4, a source application path for the radiation source can be constructed. It can be understood that the construction method of the source application path in this embodiment is also applicable to both embodiments 2 and 3 mentioned above. In other words, the source application paths of the embodiments 2 and 3 can also be simplified. On this basis, a plurality of source modules 2 can be configured for the afterloading therapy device 100 with multi-layer rotating frame 32 depending on actual needs.

Thus far, the technical solution of the present invention has been described in conjunction with the preferred implementations shown in the accompanying drawings. However, it is easy for those skilled in the art to understand that obviously the scope of protection of the present invention is not limited to these specific embodiments. Without departing from the principles of the present invention, those skilled in the art may make equivalent modifications or substitutions to the relevant technical features, and the technical solutions resulted from these modifications or substitutions will fall within the scope of protection of the present invention.

What is claimed is:

1. An afterloading therapy device, comprising:
a base;
a plurality of source modules for placing active or dummy sources;
a rotating assembly installed on the base and comprising a rotating frame, the rotating frame being equipped with a plurality of source module installation positions, and the source modules being configured to be rotatably installed on the base through the rotating assembly; and
an indexing assembly comprising an indexing plate and a plurality of indexing holes arranged on the indexing plate and corresponding to the plurality of source modules;
wherein, when observing along a source application path of the active or dummy sources, the indexing assembly is arranged on a downstream side of the rotating assembly.

2. The afterloading therapy device of claim 1, wherein the afterloading therapy device further comprises:
a convergence assembly comprising a convergence layer and a plurality of convergence pipes arranged on the convergence layer and corresponding to the plurality of indexing holes; and
a pipe connecting assembly comprising a pipe connecting plate bracket and a plurality of pipe connecting plate holes arranged on the pipe connecting plate bracket and corresponding to the plurality of convergence pipes;
wherein, the convergence assembly is located between the indexing assembly and the pipe connecting assembly.

3. The afterloading therapy device of claim 2, wherein the afterloading therapy device comprises a driving part, the driving part comprising:
a driving component; and
a support shaft arranged on the base;
the driving component being configured to drive the rotating frame to rotate through the support shaft and thus drive the source modules arranged on the rotating frame to move.

4. The afterloading therapy device of claim 3, wherein the rotating frame is a ring-shaped structure, and the driving component is configured to drive the rotating frame to rotate around its axis.

5. The afterloading therapy device of claim 4, wherein the rotating frame comprises one or more ring-shaped structures distributed radially,
wherein one or more support shafts corresponding to one or more of the rotating frames are fixedly arranged on the base,
and wherein one or more driving components corresponding to one or more of the rotating frames are configured to drive a plurality of rotating frames to rotate relative to their respective support shafts in an independent manner.

6. The afterloading therapy device of claim 5, wherein the support shaft has a cylindrical structure, and the rotating frame is arranged on an outer wall of the support shaft,
wherein the driving part further comprises a transmission mechanism, which comprises:
a gear connected to a power output end of the driving component; and
a gear ring, which is arranged on an inner or outer wall of the cylindrical structure, the gear being configured to be meshed with the gear ring, thereby causing the driving component to rotatably connect with the support shaft through the transmission mechanism.

7. The afterloading therapy device of claim 4, wherein the rotating frame comprises one or more ring-shaped structures distributed radially, and the support shaft is fixedly arranged on the rotating frame, and
in case there is one ring-shaped structure, the driving component is configured to drive the support shaft to rotate synchronously with the rotating frame;
in case there are multiple ring-shaped structures, the driving component is configured to drive the support shaft to rotate synchronously with the multiple ring-shaped structures.

8. The afterloading therapy device of claim 4, wherein the rotating frame comprises one or multiple ring-shaped structures distributed radially, and
in case there are multiple rotating frames, a plurality sets of indexing holes corresponding to the multiple ring-shaped structures are provided on the indexing plate, a plurality sets of convergence pipes corresponding to multiple ring-shaped structures are provided on the convergence layer, and a plurality sets of pipe connecting plate holes corresponding to the multiple ring-shaped structures are provided on the pipe connecting plate bracket.

9. The afterloading therapy device of claim 1, wherein the source module is arranged at the source module installation position in a detachable manner.

10. The afterloading therapy device of claim 9, wherein the rotating frame is provided with a first sliding structure arranged at a position corresponding to the source module installation position, and
the source module comprises:
a supporting structure with a second sliding structure installed thereon; and
a source storage tank, for providing active or dummy sources;
the source module being configured to be provided at the source module installation position in a push-pull manner via a sliding connection of the first sliding structure and the second sliding structure.
